(19) United States
(12) Patent Application Publication
(10) Pub. No.: US 2007/0149515 A1
(43) Pub. Date: Jun. 28, 2007

(54) 6-(2,4,6-Trifluorophenyl)-triazolopyrimidines, method for the production thereof, use thereof for controlling harmful fungi, and substances containing the same

(75) Inventors: Jordi Tormo i Blasco, Laudenbach (DE); Carsten Blettner, Mannheim (DE); Bernd Muller, Frankenthal (DE); Markus Gewehr, Kastellaun (DE); Wassilios Grammenos, Ludwigshafen (DE); Thomas Grote, Wachenheim (DE); Joachim Rheinheimer, Ludwigshafen (DE); Peter Schafer, Ottersheim (DE); Frank Schieweck, Hessheim (DE); Anja Schwogler, Mannheim (DE); Oliver Wagner, Neustadt (DE); Maria Scherer, Godramstein (DE); Siegfried Strathmann, Limburgerhof (DE); Ulrich Schofl, Bruhl (DE); Reinhard Stierl, Freinsheim (DE)

Correspondence Address: BIRCH STEWART KOLASCH & BIRCH, PO BOX 747, FALLS CHURCH, VA, 22040-0747, US

(73) Assignee: BASF Aktiengesellschaft, Ludwigshafen (DE)

(21) Appl. No.: 10/579,144

(22) PCT Filed: Nov. 18, 2004
(86) PCT No.: PCT/EP04/13063

§ 371 Date: May 15, 2006

(30) Foreign Application Priority Data
Nov. 25, 2003 (DE) 103 55 387.8

(51) Int. Cl. A01N 43/90; C07D 487/04

(52) U.S. Cl. 514/227.8; 514/259.31; 544/263; 514/234.5; 514/252.16; 544/60; 544/114

(57) ABSTRACT

6-(2,4,6-Trifluorophenyl)triazolopyrimidines of the formula I 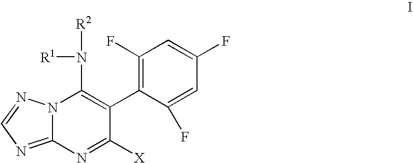 in which the substituents have the following meanings:
R¹ is alkyl, haloalkyl, cycloalkyl, halocycloalkyl, alkenyl, haloalkenyl, cycloalkenyl, alkynyl, haloalkynyl, cycloalkynyl, phenyl, naphthyl or a five- or six-membered saturated, partially unsaturated or aromatic heterocycle comprising one to four heteroatoms from the group consisting of O, N and S,
R² is hydrogen or one of the groups mentioned in R¹,
   R¹ and R² can also, together with the nitrogen atom to which they are bonded, form a five- or six-membered ring which is interrupted by an atom from the group consisting of O, N and S, and/or
   R¹ and/or R² can be substituted according to the description;
X is cyano, C₁-C₄-alkoxy, C₃-C₄-alkenyloxy, C₁-C₂-haloalkoxy or C₃-C₄-halo-alkenyloxy; process for the preparation of these compounds, preparations comprising them and their use in the control of harmful phytopathogenic fungi.

The present invention relates to 6-(2,4,6-trifluorophenyl)triazolopyrimidines of the formula I

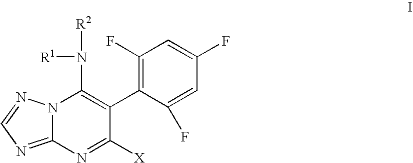

in which the substituents have the following meanings:
R¹ is C₁-C₈-alkyl, C₁-C₈-haloalkyl, C₃-C₈-cycloalkyl, C₃-C₈-halocycloalkyl, C₂-C₈-alkenyl, C₂-C₈-haloalkenyl, C₃-C₆-cycloalkenyl, C₃-C₆-halocycloalkenyl, C₂-C₈-alkynyl, C₂-C₈-haloalkynyl, phenyl, naphthyl or a five- or six-membered saturated, partially unsaturated or aromatic heterocycle comprising 1 to 4 heteroatoms from the group consisting of O, N and S,
R² is hydrogen or one of the groups mentioned in R¹,
   R¹ and R² can also, together with the nitrogen atom to which they are bonded, form a five- or six-membered heterocyclyl or heteroaryl which is bonded via N and can comprise an additional heteroatom from the group consisting of O, N and S as ring member and/or can carry one or more substituents from the group consisting of halogen, C₁-C₆-alkyl, C₁-C₆-haloalkyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, C₁-C₆-alkylene and oxy-C₁-C₃-alkylenoxy;
   R¹ and/or R² can carry one to four identical or different R^{a} groups:
   R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di(C₁-C₆-alkyl)amino, C₂-C₆-alkenyl, C₂-C₆-alkenyloxy, C₃-C₆-alkynyloxy, oxy-C₁-C₃-alkylenoxy, C₃-C₆-cycloalkyl, phenyl, naphthyl or five- or six-membered saturated, partially unsaturated or aromatic heterocycle comprising one to four heteroatoms from the group consisting of O, N and S,
      these aliphatic, alicyclic or aromatic groups for their part being able to be partially or completely halogenated;
X is cyano, C₁-C₄-alkoxy, C₃-C₄-alkenyloxy, C₁-C₂-haloalkoxy or C₃-C₄-halo-alkenyloxy.

In addition, the invention relates to a process for the preparation of these compounds, preparations comprising them and their use in the control of harmful phytopathogenic fungi.

5-Halo-6-(2,4,6-trifluorophenyl)triazolopyrimidines are generally known from WO 98/46607. 5-Cyano- and 5-alkoxytriazolopyrimidines are revealed in WO 02/083677. Triazolopyrimidines with optically active amino substituents in the 7-position are generally proposed in WO 02/38565.

The compounds disclosed in the aforesaid documents are suitable for the control of harmful fungi.

However, their action is not always in every respect completely satisfactory. It is an object of the present invention to provide compounds which have an improved action and/or a broadened spectrum of activity.

We have found that this object is achieved by the compounds defined at the start. Furthermore, a process for their preparation, preparations comprising them and methods for the control of harmful fungi with the use of the compounds I have been found.

The compounds according to the invention are distinguished from those disclosed in the aforementioned document by the substitution in the 5-position of the triazolopyrimidine backbone.

The compounds of the formula I have, in comparison with the known compounds, an increased effectiveness or a broadened spectrum of activity against harmful fungi.

The compounds according to the invention can be obtained in various ways. They are advantageously obtained starting from the 5-halo-6-(2,4,6-trifluorophenyl)triazolo-pyrimidines of the formula II known from WO 98/46607 by reaction with compounds M-X (formula III). Compounds III represent, depending on the meaning of the X group to be introduced, an inorganic cyanide or an alkoxide. The reaction is advantageously carried out in the presence of an inert solvent. The cation M in the formula III is of little significance; for practical reasons, ammonium, tetraalkylammonium or alkali metal or alkaline earth metal salts are usually preferred.

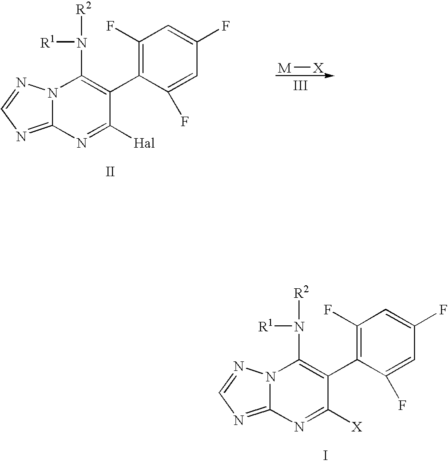

The reaction temperature is usually from 0 to 120° C., preferably from 10 to 40° C. [cf. J. Heterocycl. Chem., Vol. 12, pp. 861-863 (1975)].

If R² represents hydrogen, a cleavable protective group is advantageously introduced before reaction with III [cf. Greene, Protective Groups in Organic Chemistry, J. Wiley & Sons (1981)].

Suitable solvents comprise ethers, such as dioxane, diethyl ether and, preferably, tetrahydrofuran, halogenated hydrocarbons, such as dichloromethane, aromatic hydrocarbons, such as toluene, or acetonitrile.

The reaction mixtures are worked up conventionally, e.g. by mixing with water, separating the phases and possibly chromatographic purification of the crude products. Some of the intermediates and final products are obtained in the form of colorless or slightly brownish viscous oils which, under reduced pressure and at moderately elevated temperature, are freed from or purified of volatile constituents. Provided that the intermediates and final products are obtained as solids, the purification can also take place by recrystallization or trituration.

If individual compounds I are not accessible by the routes described above, they can be prepared by derivatization of other compounds I.

If mixtures of isomers are obtained in the synthesis, a separation is generally not absolutely essential, however, since the individual isomers can sometimes be converted into one another during the workup for the application or in the application (e.g. under the action of light, acid or base). Appropriate conversions can also take place after the application, for example, with the treatment of plants, in the treated plants or in the harmful fungi to be controlled.

Collective terms were used in the definitions of the symbols given in the above formulae, which collective terms are generally representative of the following substituents:

halogen: fluorine, chlorine, bromine and iodine;

alkyl: saturated, straight-chain or branched hydrocarbon radicals with 1 to 4, 6 or 8 carbon atoms, e.g. C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

haloalkyl: straight-chain or branched alkyl groups with 1 to 2, 4, 6 or 8 carbon atoms (as mentioned above), in which the hydrogen atoms in these groups can be partially or completely replaced by halogen atoms as mentioned above: in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;

alkenyl: unsaturated, straight-chain or branched hydrocarbon radicals with 2 to 4, 6, 8 or 10 carbon atoms and one or two double bonds in any position, e.g. C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-3pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-di-methyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

haloalkenyl: unsaturated, straight-chain or branched hydrocarbon radicals with 2 to 8 carbon atoms and one or two double bonds in any position (as mentioned above), in which the hydrogen atoms in these groups can be partially or completely replaced by halogen atoms as mentioned above, in particular fluorine, chlorine and bromine;

alkynyl: straight-chain or branched hydrocarbon groups with 2 to 4, 6 or 8 carbon atoms and one or two triple bonds in any position, e.g. C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl4-pentynyl, 2-methyl-3-pentynyl, 2-methyl4-pentynyl, 3-methyl-1-pentynyl, 3-methyl⁴-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

cycloalkyl: saturated, mono- or bicyclic hydrocarbon groups with 3 to 6 or 8 carbon ring members, e.g. C₃-C₈-cycloalkyl, such as cyclopropyl, cyclobutyl, cyclopentyl, cyclohexyl, cycloheptyl and cyclooctyl;

five- or six-membered saturated, partially unsaturated or aromatic heterocycle comprising one to four heteroatoms from the group consisting of O, N and S:
5- or 6-membered heterocyclyl comprising one to three nitrogen atoms and/or one oxygen or sulfur atom or one or two oxygen and/or sulfur atoms, e.g. 2-tetrahydro-furanyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 2-pyrrolin-2-yl, 2-pyrrolin-3-yl, 3-pyrrolin-2-yl, 3-pyrrolin-3-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexa-hydropyrimidinyl and 2-piperazinyl;
5-membered heteroaryl comprising one to four nitrogen atoms or one to three nitrogen atoms and one sulfur or oxygen atom: 5-ring heteroaryl groups which, in addition to carbon atoms, can comprise one to four nitrogen atoms or one to three nitrogen atoms and one sulfur or oxygen atom as ring members, e.g. 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl and 1,3,4-triazol-2-yl;
6-membered heteroaryl comprising one to three or one to four nitrogen atoms: 6-ring heteroaryl groups which, in addition to carbon atoms, can comprise one to three or one to four nitrogen atoms as ring members, e.g. 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl;

alkylene: saturated, straight-chain or branched hydrocarbon radicals with 1 to 4 or 6 carbon atoms which are bonded to the backbone via a double bond, e.g. ═CH₂, ═CH—CH₃, ═CH—CH₂—CH₃;

oxyalkylenoxy: unbranched divalent chains formed from 1 to 3 CH₂ groups in which both valencies are bonded to the backbone via an oxygen atom, e.g. OCH₂O, OCH₂CH₂O and OCH₂CH₂CH₂O.

The (R)- and (S)-isomers and the racemates of compounds of the formula I which have chiral centers are included in the present invention.

In view of the intended use of the triazolopyrimidines of the formula I, the following meanings of the substituents, in each case alone or in combination, are especially preferred:

Preference is given to compounds I in which R¹ is C₁-C₄-alkyl, C₂-C₆-alkenyl or C₁-C₈-haloalkyl.

Preference is given to compounds I in which R¹ is a group A:

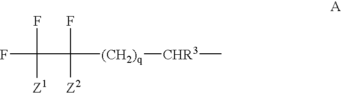

in which
Z¹ is hydrogen, fluorine or C₁-C₆-fluoroalkyl,
Z² is hydrogen or fluorine, or Z¹ and Z² together form a double bond;
q is 0 or 1; and
R³ represents hydrogen or methyl.

In addition, preference is given to compounds I in which R¹ is C₃-C₆-cycloalkyl which can be substituted by C₁-C₄-alkyl.

Particular preference is given to compounds I in which R² represents hydrogen.

Preference is similarly given to compounds I in which R² is methyl or ethyl.

If R¹ and/or R² comprise haloalkyl or haloalkenyl groups with a chiral center, the (S)-isomers are preferred for these groups. In the case of halogen-free alkyl or alkenyl groups with a chiral center in R¹ or R², the (R)-configurated isomers are preferred.

A preferred embodiment of the invention relates to compounds of the formula I.1:

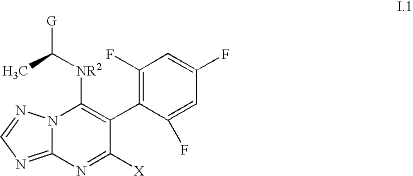

wherein
G represents C₂-C₆-alkyl, in particular ethyl, n-propyl, isopropyl, n-butyl, sec-butyl or tert-butyl, C₁-C₄-alkoxymethyl, in particular ethoxymethyl, or C₃-C₆-cycloalkyl, in particular cyclopentyl or cyclohexyl;
R² represents hydrogen or methyl; and
X represents cyano, methoxy or ethoxy.

An additional preferred embodiment of the invention relates to compounds of the formula I.2:

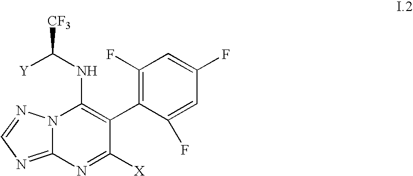

wherein Y is hydrogen or C₁-C₄-alkyl, in particular methyl and ethyl, and X is cyano, methoxy or ethoxy.

An additional preferred embodiment of the invention relates to compounds wherein R¹ and R², together with the nitrogen atom to which they are bonded, form a five- or six-membered heterocyclyl or heteroaryl which is bonded via N and can comprise an additional heteroatom from the group consisting of O, N and S as ring member and/or can carry one or more substituents from the group consisting of halogen, C₁-C₆-alkyl,

C₁-C₆-haloalkyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, C₁-C₆-alkylene and oxy-C₁-C₃-alkylenoxy. These compounds correspond in particular to formula I.3:

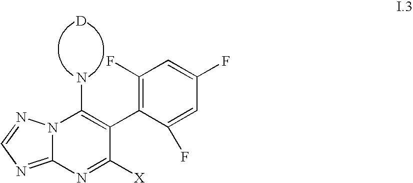

wherein
D together with the nitrogen atom, forms a five- or six-membered heterocyclyl or heteroaryl which is bonded via N and can comprise an additional heteroatom from the group consisting of O, N and S as ring member and/or can carry one or more substituents from the group consisting of halogen, C₁-C₄-alkyl, C₁-C₄-alkoxy and C₁-C₂-haloalkyl; and
X represents cyano, methoxy or ethoxy.

Preference is furthermore given to compounds I wherein R¹ and R², together with the nitrogen atom to which they are bonded, form a piperidinyl, morpholinyl or thio-morpholinyl ring, in particular a piperidinyl ring, which, if appropriate, is substituted by one to three halogen, C₁-C₄-alkyl or C₁-C₄-haloalkyl groups. Particular preference is given to the compounds wherein R¹ and R², together with the nitrogen atom to which they are bonded, form a 4-methylpiperidine ring.

An additional preferred object of the invention are compounds I wherein R¹ and R², together with the nitrogen atom to which they are bonded, form a pyrazole ring which, if appropriate, is substituted by one or two halogen, C₁-C₄-alkyl or C₁-C₄-haloalkyl groups, in particular by 3,5-dimethyl or 3,5-di(trifluoromethyl).

In addition, particular preference is also given to compounds of the formula I wherein R¹ represents CH(CH₃)—CH₂CH₃, CH(CH₃)—CH(CH₃)₂, CH(CH₃)—C(CH₃)₃, CH(CH₃)—CF₃, CH₂C(CH₃)═CH₂, CH₂CH═CH₂, cyclopentyl or cyclohexyl; R² represents hydrogen or methyl; or R¹ and R² together represent —(CH₂)₂CH(CH₃)(CH₂)₂—, —(CH₂)₂CH(CF₃)(CH₂)₂— or —(CH₂)₂O(CH₂)₂—.

Particular preference is furthermore given to compounds I wherein X represents cyano, methoxy or ethoxy, in particular cyano or methoxy.

Particular preference is given, in view of their use, to the compounds I compiled in the following tables. The groups mentioned in the tables for a substituent additionally represent, considered per se, independently of the combination in which they are mentioned, a particularly preferred form of the substituent in question.

Table 1

Compounds of the formula I in which X represents cyano and the combination of R¹ and R² for a compound corresponds in each case to a row of table A

Table 2

Compounds of the formula I in which X represents methoxy and the combination of R¹ and R² for a compound corresponds in each case to a row of table A

Table 3

Compounds of the formula I in which X represents ethoxy and the combination of R¹ and R² for a compound corresponds in each case to a row of table A

TABLE A

| No. | R¹ | R² |
|---|---|---|
| A-1 | CH₃ | H |
| A-2 | CH₃ | CH₃ |
| A-3 | CH₂CH₃ | H |
| A-4 | CH₂CH₃ | CH₃ |
| A-5 | CH₂CH₃ | CH₂CH₃ |
| A-6 | CH₂CF₃ | H |
| A-7 | CH₂CF₃ | CH₃ |
| A-8 | CH₂CF₃ | CH₂CH₃ |
| A-9 | CH₂CCI₃ | H |
| A-10 | CH₂CCI₃ | CH₃ |
| A-11 | CH₂CCI₃ | CH₂CH₃ |
| A-12 | CH₂CH₂CH₃ | H |
| A-13 | CH₂CH₂CH₃ | CH₃ |
| A-14 | CH₂CH₂CH₃ | CH₂CH₃ |
| A-15 | CH₂CH₂CH₃ | CH₂CH₂CH₃ |
| A-16 | CH(CH₃)₂ | H |
| A-17 | CH(CH₃)₂ | CH₃ |
| A-18 | CH(CH₃)₂ | CH₂CH₃ |
| A-19 | CH₂CH₂CH₂CH₃ | H |
| A-20 | CH₂CH₂CH₂CH₃ | CH₃ |
| A-21 | CH₂CH₂CH₂CH₃ | CH₂CH₃ |
| A-22 | CH₂CH₂CH₂CH₃ | Ch₂CH₂CH₃ |
| A-23 | CH₂CH₂CH₂CH₃ | CH₂CH₂CH₂CH₃ |
| A-24 | (±)CH(CH₃₎—CH₂CH₃ | H |
| A-25 | (±)CH(CH₃₎—CH₂CH₃ | CH₃ |
| A-26 | (±)CH(CH₃₎—CH₂CH₃ | CH₂CH₃ |
| A-27 | (S) CH(CH₃)—CH₂CH₃ | H |
| A-28 | (S) CH(CH₃)—CH₂CH₃ | CH₃ |
| A-29 | (S) CH(CH₃)—CH₂CH₃ | CH₂CH₃ |
| A-30 | (R) CH(CH₃)—CH₂CH₃ | H |
| A-31 | (R) CH(CH₃)—CH₂CH₃ | CH₃ |
| A-32 | (R) CH(CH₃)—CH₂CH₃ | CH₂CH₃ |
| A-33 | (±)CH(CH₃)—CH(CH₃)₂ | H |
| A-34 | (±)CH(CH₃)—CH(CH₃)₂ | CH₃ |
| A-35 | (±)CH(CH₃)—CH(CH₃)₂ | CH₂CH₃ |
| A-36 | (S) CH(CH₃)—CH(CH₃)₂ | H |
| A-37 | (S) CH(CH₃)—CH(CH₃)₂ | CH₃ |
| A-38 | (S) CH(CH₃)—CH(CH₃)₂ | CH₂CH₃ |
| A-39 | (R) CH(CH₃)—CH(CH₃)₂ | H |
| A-40 | (R) CH(CH₃)—CH(CH₃)₂ | CH₃ |
| A-41 | (R) CH(CH₃)—CH(CH₃)₂ | CH₂CH₃ |
| A-42 | (±)CH(CH₃)—C(CH₃)₃ | H |
| A-43 | (±)CH(CH₃)—C(CH₃)₃ | CH₃ |
| A-44 | (±)CH(CH₃)—C(CH₃)₃ | CH₂CH₃ |
| A-45 | (S) CH(CH₃)—C(CH₃)₃ | H |
| A-46 | (S) CH(CH₃)—C(CH₃)₃ | CH₃ |
| A-47 | (S) CH(CH₃)—C(CH₃)₃ | CH₂CH₃ |
| A-48 | (R) CH(CH₃)—C(CH₃)₃ | H |
| A-49 | (R) CH(CH₃)—C(CH₃)₃ | CH₃ |
| A-50 | (R) CH(CH₃)—C(CH₃)₃ | CH₂CH₃ |
| A-51 | (±)CH(CH₃)—CF₃ | H |
| A-52 | (±)CH(CH₃)—CF₃ | CH₃ |
| A-53 | (±)CH(CH₃)—CF₃ | CH₂CH₃ |
| A-54 | (S) CH(CH₃)—CF₃ | H |
| A-55 | (S) CH(CH₃)—CF₃ | CH₃ |
| A-56 | (S) CH(CH₃)—CF₃ | CH₂CH₃ |
| A-57 | (R) CH(CH₃)—CF₃ | H |
| A-58 | (R) CH(CH₃)—CF₃ | CH₃ |
| A-59 | (R) CH(CH₃)—CF₃ | CH₂CH₃ |
| A-60 | (±)CH(CH₃)—CCI₃ | H |
| A-61 | (±)CH(CH₃)—CCI₃ | CH₃ |
| A-62 | (±)CH(CH₃)—CCI₃ | CH₂CH₃ |
| A-63 | (S) CH(CH₃)—CCI₃ | H |
| A-64 | (S) CH(CH₃)—CCI₃ | CH₃ |
| A-65 | (S) CH(CH₃)—CCI₃ | CH₂CH₃ |
| A-66 | (R) CH(CH₃)—CCI₃ | H |
| A-67 | (R) CH(CH₃)—CCI₃ | CH₃ |
| A-68 | (R) CH(CH₃)—CCI₃ | CH₂CH₃ |
| A-69 | CH₂CF₂CF₃ | H |
| A-70 | CH₂CF₂CF₃ | CH₃ |
| A-71 | CH₂CF₂CF₃ | CH₂CH₃ |
| A-72 | CH₂(CF₂)₂CF₃ | H |
| A-73 | CH₂(CF₂)₂CF₃ | CH₃ |
| A-74 | CH₂(CF₂)₂CF₃ | CH₂CH₃ |
| A-75 | CH₂C(CH₃)═CH₂ | H |
| A-76 | CH₂C(CH₃)═CH₂ | CH₃ |
| A-77 | CH₂C(CH₃)═CH₂ | CH₂CH₃ |
| A-78 | CH₂CH═CH₂ | H |
| A-79 | CH₂CH═CH₂ | CH₃ |
| A-80 | CH₂CH═CH₂ | CH₂CH₃ |
| A-81 | CH₂—C═CH | H |
| A-82 | CH₂—C═CH | CH₃ |
| A-83 | CH₂—C═CH | CH₂CH₃ |
| A-84 | Cyclopentyl | H |
| A-85 | Cyclopentyl | CH₃ |
| A-86 | Cyclopentyl | CH₂CH₃ |
| A-87 | Cyclohexyl | H |
| A-88 | Cyclohexyl | CH₃ |
| A-89 | Cyclohexyl | CH₂CH₃ |
| A-90 | CH₂—C₆H₅ | H |
| A-91 | CH₂—C₆H₅ | CH₃ |
| A-92 | CH₂—C₆H₅ | CH₂CH₃ |

| | |
|---|---|
| A-93 | —(CH₂)₂CH═CHCH₂— |
| A-94 | —(CH₂)₂C(CH₃)═CHCH₂— |
| A-95 | —(CH₂)₂CH(CH₃)(CH₂)₂— |
| A-96 | —(CH₂)₃CHFCH₂— |
| A-97 | —(CH₂)₂CHF(CH₂)₂— |
| A-98 | —CH₂CHF(CH₂)₃— |
| A-99 | —(CH₂)₂CH(CF₃)(CH₂)₂— |
| A-100 | —(CH₂)₂O(CH₂)₂— |
| A-101 | —(CH₂)₂S(CH₂)₂— |
| A-102 | —(CH₂)₅— |
| A-103 | —(CH₂)₄— |
| A-104 | —CH₂CH═CHCH₂— |
| A-105 | —CH(CH₃)(CH₂)₃— |
| A-106 | —CH₂CH(CH₃)(CH₂)₂— |
| A-107 | —CH(CH₃)—(CH₂)₂—CH(CH₃)— |
| A-108 | —CH(CH₃)—(CH₂)₄— |
| A-109 | —CH₂—CH(CH₃)—(CH₂)₃— |
| A-110 | —(CH₂)—CH(CH₃)—CH₂—CH(CH₃)—CH₂— |
| A-111 | —CH(CH₂CH₃)—(CH₂)₄— |
| A-112 | —(CH₂)₂—CHOH—(CH₂)₂— |
| A-113 | —(CH₂)—CH═CH—(CH₂)₂— |
| A-114 | —(CH₂)₆— |
| A-115 | —CH(CH₃)—(CH₂)₅— |
| A-116 | —(CH₂)₂—N(CH₃)—(CH₂)₂— |
| A-117 | —N═CH—CH═CH— |
| A-118 | —N═C(CH₃)—CH═C(CH₃)— |
| A-119 | —N═C(CF₃)—CH═C(CF₃)— |

The compounds I are suitable as fungicides. They are distinguished by an outstanding effectiveness against a broad spectrum of phytopathogenic fungi, especially from the classes of the Ascomycetes, Deuteromycetes, Oomycetes and Basidiomycetes. Some are systemically effective and they can be used in plant protection as foliar and soil fungicides.

They are particularly important in the control of a multitude of fungi on various cultivated plants, such as wheat, rye, barley, oats, rice, maize, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on fruit and vegetables,
Bipolaris and Drechslera species on cereals, rice and lawns,
Blumeria graminis (powdery mildew) on cereals,
Botrytis cinerea (gray mold) on strawberries, vegetables, ornamental plants and grapevines,
Erysiphe cichoracearum and Sphaerotheca fuliginea on cucurbits,
Fusarium and Verticillium species on various plants,
Mycosphaerella species on cereals, bananas and peanuts,
Phytophthora infestans on potatoes and tomatoes,
Plasmopara viticola on grapevines,
Podosphaera leucotricha on apples,
Pseudocercosporella herpotrichoides on wheat and barley,
Pseudoperonospora species on hops and cucumbers,
Puccinia species on cereals,
Pyricularia oryzae on rice,
Rhizoctonia species on cotton, rice and lawns,
Septoria tritici and Stagonospora nodorum on wheat,
Uncinula necator on grapevines,
Ustilago species on cereals and sugar cane, and
Venturia species (scab) on apples and pears.

The compounds I are also suitable for controlling harmful fungi, such as Paecilomyces variotii, in the protection of materials (e.g. wood, paper, paint dispersions, fibers or fabrics) and in the protection of stored products.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, amounts of active compound of 1 to 1000 g/100 kg, preferably 5 to 100 g per 100 kilograms, of seed are generally necessary.

When used in the protection of materials or stored products, the amount of active compound applied depends on the kind of application area and on the effect desired. Amounts customarily applied in the protection of materials are, for example, 0.001 g to 2 kg, preferably 0.005 g to 1 kg, of active compound per cubic meter of treated material.

The compounds I can be converted to the usual formulations, e.g. solutions, emulsions, suspensions, dusts, powders, pastes and granules. The application form depends on the respective use intended; it should always guarantee a fine and uniform distribution of the compound according to the invention.

The formulations are prepared in a known way, e.g. by extending the active compound with solvents and/or carriers, if desired using emulsifiers and dispersants. Suitable solvents/auxiliaries for this are in the main:
water, aromatic solvents (e.g. Solvesso products, xylene), paraffins (e.g. petroleum fractions), alcohols (e.g. methanol, butanol, pentanol, benzyl alcohol), ketones (e.g. cyclohexanone, gamma-butyrolactone), pyrrolidones (NMP, NOP), acetates (glycol diacetate), glycols, fatty acid dimethylamides, fatty acids and fatty acid esters. Mixtures of solvents can also be used in principle,
carriers, such as ground natural minerals (e.g. kaolins, clays, talc, chalk) and ground synthetic ores (e:g. highly dispersed silicic acid, silicates); emulsifiers, such as nonionic and anionic emulsifiers (e.g. polyoxyethylene fatty alcohol ethers, alkylsulfonates and arylsulfonates) and dispersants, such as lignosulfite waste liquors and methylcellulose.

Alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers are used as surfactants; condensation products of sulfonated naphthalene and naphthalene derivatives with formaldehyde, condensation products of naphthalene or of naphthalenesulfonic acid with phenol and formaldehyde, polyoxyethylene octylphenol ethers, ethoxylated isooctylphenol, octylphenol and nonylphenol, alkylphenol polyglycol ethers, tributylphenyl polyglycol ethers, tristearylphenyl polyglycol ethers, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose are also suitable surfactants.

Petroleum fractions having medium to high boiling points, such as kerosene or diesel fuel, furthermore coal tar oils, and oils of vegetable or animal origin, aliphatic, cyclic and aromatic hydrocarbons, e.g. toluene, xylene, paraffin, tetrahydronaphthalene, alkylated naphthalenes or derivatives thereof, methanol, ethanol, propanol, butanol, cyclohexanol, cyclohexanone, isophorone or highly polar solvents, e.g. dimethyl sulfoxide, N-methylpyrrolidone or water, are suitable for the preparation of directly sprayable solutions, emulsions, pastes or oil dispersions.

Powders, preparations for broadcasting and dusts can be prepared by mixing or mutually grinding the active substances with a solid carrier.

Granules, e.g. coated granules, impregnated granules and homogeneous granules, can be prepared by binding the active compounds to solid carriers. Solid carriers are, e.g., mineral earths, such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, e.g., ammonium sulfate, ammonium phosphate, ammonium nitrate or ureas, and plant products, such as cereal meal, tree bark meal, wood meal and nutshell meal, cellulose powders and other solid carriers.

The formulations generally comprise between 0.01 and 95% by weight, preferably between 0.1 and 90% by weight, of the active compound. The active compounds are employed therein in a purity of 90% to 100%, preferably 95% to 100% (according to the NMR spectrum).

Examples for Formulations are:

1. Products for Diluting in Water

A Water-Soluble Concentrates (SL)

10 parts by weight of a compound according to the invention are dissolved in water or a water-soluble solvent. Alternatively, wetting agents or other auxiliaries are added. The active compound dissolves on diluting in water.

B Dispersible Concentrates (DC)

20 parts by weight of a compound according to the invention are dissolved in cyclohexanone with addition of a dispersant, e.g. polyvinylpyrrolidone. A dispersion is formed on diluting in water.

C Emulsifiable Concentrates (EC)

15 parts by weight of a compound according to the invention are dissolved in xylene with addition of calcium dodecylbenzenesulfonate and castor oil ethoxylate (each 5%). An emulsion is formed on diluting in water.

D Emulsions (EW, EO)

40 parts by weight of a compound according to the invention are dissolved in xylene with addition of calcium dodecylbenzenesulfonate and castor oil ethoxylate (each 5%). This mixture is introduced into water using an emulsifying apparatus (UltraTurrax) and is converted to a homogeneous emulsion. An emulsion is formed on diluting in water.

E Suspensions (SC, OD)

20 parts by weight of a compound according to the invention are reduced to a fine active compound suspension in an agitator ball mill with addition of dispersants and wetting agents and water or an organic solvent. A stable suspension of the active compound is formed on diluting in water.

F Water-Dispersible and Water-Soluble Granules (WG, SG)

50 parts by weight of a compound according to the invention are finely milled with addition of dispersants and wetting agents and, using industrial devices (e.g. extrusion, spray tower, fluid bed), are prepared as water-dispersible or water-soluble granules. A stable dispersion or solution of the active compound is formed on diluting in water.

G Water-Dispersible and Water-Soluble Powders (WP, SP)

75 parts by weight of a compound according to the invention are milled in a rotor-stator mill with addition of dispersants and wetting agents and also of silica gel. A stable dispersion or solution of the active compound is formed on diluting in water.

2. Products for Direct Application

H Dustable Powders (DP)

5 parts by weight of a compound according to the invention are finely milled and thoroughly mixed with 95% of finely divided kaolin. A dust is thereby obtained.

I. Granules (GR, FG, GG, MG)

0.5 part by weight of a compound according to the invention is finely milled and combined with 95.5% of carriers. Standard processes in this connection are extrusion, spray drying or fluid bed. A granule for direct application is thereby obtained.

J ULV Solutions (UL)

10 parts by weight of a compound according to the invention are dissolved in an organic solvent, e.g. xylene. A product for direct application is thereby obtained.

The active compounds can be used as such, in the form of their formulations or of the application forms prepared therefrom, e.g. in the form of directly sprayable solutions, powders, suspensions or dispersions, emulsions, oil dispersions, pastes, dusts, preparations for broadcasting or granules, by spraying, atomizing, dusting, broadcasting or watering. The application forms depend entirely on the intended uses; they should always guarantee the finest possible dispersion of the active compounds according to the invention.

Aqueous use forms can be prepared from emulsifiable concentrates, pastes or wettable powders (spray powders, oil dispersions) by addition of water. To prepare emulsions, pastes or oil dispersions, the substances can be homogenized in water, as such or dissolved in an oil or solvent, by means of wetting agents, tackifiers, dispersants or emulsifiers. However, concentrates comprising active substance, wetting agent, tackifier, dispersant or emulsifier and possibly solvent or oil can also be prepared, which concentrates are suitable for dilution with water.

The concentrations of active compound in the ready-for-use preparations can be varied within relatively wide ranges. In general, they are between 0.0001 and 10%, preferably between 0.01 and 1%.

The active compounds can also be used with great success in the ultra low volume (ULV) process, it being possible to apply formulations with more than 95% by weight of active compound or even the active compound without additives.

Oils of various types, wetting agents, adjuvants, herbicides, fungicides, other pesticides and bactericides can be added to the active compounds, if appropriate also not until immediately before use (tank mix). These agents can be added to the preparations according to the invention in a weight ratio of 1:10 to 10:1.

The preparations according to the invention can, in the application form as fungicides, also be present together with other active compounds, e.g. with herbicides, insecticides, growth regulators, fungicides or also with fertilizers. On mixing the compounds I or the preparations comprising them in the application form as fungicides with other fungicides, in many cases an expansion of the fungicidal spectrum of activity is obtained.

The following list of fungicides, with which the compounds according to the invention can be used in conjunction, is intended to illustrate the possible combinations but not to limit them:
acylalanines, such as benalaxyl, metalaxyl, ofurace or oxadixyl,
amine derivatives, such as aldimorph, dodine, dodemorph, fenpropimorph, fenpropidin, guazatine, iminoctadine, spiroxamine or tridemorph,
anilinopyrimidines, such as pyrimethanil, mepanipyrim or cyprodinil,
antibiotics, such as cycloheximide, griseofulvin, kasugamycin, natamycin, polyoxin or streptomycin,
azoles, such as bitertanol, bromoconazole, cyproconazole, difenoconazole, diniconazole, epoxiconazole, fenbuconazole, fluquinconazole, flusilazole, hexaconazole, imazalil, metconazole, myclobutanil, penconazole, propiconazole, prochloraz, prothioconazole, tebuconazole, triadimefon, triadimenol, triflumizole or triticonazole,
dicarboximides, such as iprodione, myclozolin, procymidone or vinclozolin,
dithiocarbamates, such as ferbam, nabam, maneb, mancozeb, metam, metiram, propineb, polycarbamate, thiram, ziram or zineb,
heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamide, thiophanate-methyl, tiadinil, tricyclazole or triforine,
copper fungicides, such as Bordeaux mixture, copper acetate, copper oxychloride or basic copper sulfate,
nitrophenyl derivatives, such as binapacryl, dinocap, dinobuton or nitrothal-isopropyl,
phenylpyrroles, such as fenpiclonil or fludioxonil,
sulfur,
other fungicides, such as acibenzolar-S-methyl, benthiavalicarb, carpropamid, chlorothalonil, cyflufenamid, cymoxanil, dazomet, diclomezine, diclocymet, diethofencarb, edifenphos, ethaboxam, fenhexamid, fentin acetate, fenoxanil, ferimzone, fluazinam, fosetyl, fosetyl-aluminum, iprovalicarb, hexachlorobenzene, metrafenone, pencycuron, propamocarb, phthalide, tolclofos-methyl, quintozene or zoxamide,
strobilurins, such as azoxystrobin, dimoxystrobin, fluoxastrobin, kresoxim-methyl, metominostrobin, orysastrobin, picoxystrobin, pyraclostrobin or trifloxystrobin,
sulfenic acid derivatives, such as captafol, captan, dichlofluanid, folpet or tolylfluanid,
cinnamamides and analogous compounds, such as dimethomorph, flumetover or flumorph.

### SYNTHESIS EXAMPLES

The procedures described in the following synthesis examples were used to prepare further compounds I by appropriate modification of the starting compounds. The compounds thus obtained are listed in the following table, together with physical data.

### Example 1

### Preparation of 5-methoxy-6-(2,4,6-trifluorophenyl)-7-(4-methylpiperidinyl)[1,2,4]triazolo[1,5a]pyrimidine

A solution of 0.6 g (1.5 mmol) of 5-chloro-6-(2,4,6-trifluorophenyl)-7-(4-methylpiperidinyl)[1,2,4]triazolo[1,5a]pyrimidine (cf. WO 98/46607) in 10 ml of methanol was treated with 0.32 g (1.8 mmol) of 30% sodium methoxide solution and was stirred overnight at 20-25° C. After diluting the reaction mixture with dilute hydrochloric acid, extracting the aqueous phase with methyl t-butyl ether (MTBE) and separating the phases, the combined organic phases were concentrated. The residue was purified using MPLC over silica gel RP-18 with acetonitrile/water mixtures. 0.3 g of the title compound was obtained as pale crystalline substance (M.p. 172-174° C.).

¹H NMR (CDCl₃, δ in ppm): 8.25 (s,1 H), 6.8 (t, 2H), 4.0 (s, 3H), 3.6 (d, 2H), 2.8 (m, 2H), 1.65 (m, 2H), 1.55 (m, 1H), 1.35 (m, 2H), 1.0 (d, 3H)

### Example 2

### Preparation of 5-cyano-6-(2,4,6-trifluorophenyl)-7-(4-methylpiperidinyl)[1,2,4]triazolo[1,5a]pyrimidine

A solution of 1 g (2.5 mmol) of 5-chloro-6-(2,4,6-trifluorophenyl)-7-(4-methylpiperidinyl)[1,2,4]triazolo[1,5a]pyrimidine (WO 98/46607) in 20 ml of acetonitrile was treated with 1.9 g (6.5 mmol) of tetrabutylammonium cyanide and was stirred overnight at 20-25° C. After diluting the reaction mixture with dilute hdrochloric acid, extracting the aqueous phase with methyl t-butyl ether (MTBE) and separating the phases, the combined organic phases were concentrated. The residue was purified using MPLC over silica gel RP-18 with acetonitrile/water mixtures. 0.3 g of the title compound was obtained as pale crystalline substance (M.p. 205-206° C.).

¹H NMR (CDCl₃, δ in ppm): 8.5 (s, 1H), 7.9 (t, 2H), 3.8 (d, 2H), 2.9 (m, 2H), 1.7 (M, 2H), 1.6 (m,1 H), 1.35 (m, 2H), 1.0 (d, 3H)

TABLE I
Compounds of the formula I

| | | | | Phys. Data |
|---|---|---|---|---|
| | | | | (¹H NMR [δ ppm]; |
| No. | R¹ | R² | X | M.p. [° C.]) |
| I-1 | (S) CH(CH₃)—CF₃ | H | CN | 8.55 (s, 1H), 6.95 (m, |
| | | | | 2H), 1.5 (d, 3H) |
| I-2 | —(CH₂)₂CH(CH₃)(CH₂)₂— | | CN | 205-206 |
| I-3 | (S) CH(CH₃)—CF₃ | H | OCH₃ | 172-174 |
| I-4 | CH(CH₃)₂ | H | OCH₃ | 155 |

| | | | |
|---|---|---|---|
| I-5 | —(CH₂)₂CH(CH₃)(CH₂)₂— | OCH₃ | 175-177 |

Examples for the action against harmful fungi

The fungicidal action of the compounds of the formula I can be demonstrated by the following tests:

The active compounds were prepared separately or together as a stock solution with 0.25% by weight of active compound in acetone or DMSO. 1% by weight of the emulsifier Uniperol® EL (wetting agent with an emulsifying and dispersing action based on ethoxylated alkylphenols) was added to this solution and appropriately diluted with water to the desired concentration.

Use example 1—Activity in protective application against powdery mildew on cucumber leaves caused by Sphaerotheca fuliginea

Leaves of pot-grown cucumber seedlings of the variety “Chinese Snake” in the cotyledon stage were sprayed to runoff point with an aqueous suspension in the active compound concentration given below. 20 hours after the spray coating had dried on, the plants were inoculated with an aqueous suspension of spores of powdery mildew of cucumber (Sphaerotheca fuliginea). The plants were subsequently cultivated for 7 days in a greenhouse at temperatures of between. 20 and 24° C. and a relative atmospheric humidity of 60 to 80%. The extent of mildew development was then determined visually in % of infection of the cotyledon area.

In this test, the plants treated with 63 ppm of the compounds I-1, I-2, I-3 or I-5 showed no infection, while the untreated plants were 100% infected.

Use example 2—Activity in 1 day protective application against net blotch of barley caused by Pyrenophora teres

Leaves of pot-grown barley seedlings were sprayed to runoff point with an aqueous suspension in the active compound concentration given below. 24 hours after the spray coating had dried on, the test plants were inoculated with an aqueous suspension of spores of Pyrenophora [syn. Drechslera] teres, the causative agent of net blotch. The test plants were subsequently placed in a greenhouse at temperatures of between 20 and 24° C. and a relative atmospheric humidity of 95 to 100%. After 6 days, the extent of development of the disease was determined visually in % of infection of the total leaf area.

In this test, the plants treated with 63 ppm of the compounds I-1, I-2, I-3 or I-5 showed no infection, while the untreated plants were 90% infected.

1. A 6-(2,4,6-trifluorophenyl)triazolopyrimidine of the formula I 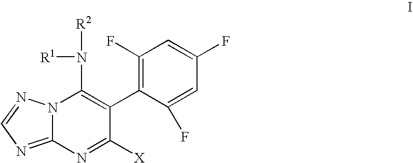
in which the substituents have the following meanings:
R¹ is C₁-C₈-alkyl, C₁-C₈-haloalkyl, C₃-C₈-cycloalkyl, C₃-C₈-halocycloalkyl, C₂-C₈-alkenyl, C₂-C₈-haloalkenyl, C₃-C₆-cycloalkenyl, C₃-C₆-halocycloalkenyl, C₂-C₈-alkynyl, C₂-C₈-haloalkynyl, phenyl, naphthyl or a five- or six-membered saturated, partially unsaturated or aromatic heterocycle comprising 1 to 4 heteroatoms from the group consisting of O, N and S,
R² is hydrogen or one of the groups mentioned in R¹,
R¹ and R² can also, together with the nitrogen atom to which they are bonded, form a five- or six-membered heterocyclyl or heteroaryl which is bonded via N and can comprise an additional heteroatom from the group consisting of O, N and S as ring member and/or can carry one or more substituents from the group consisting of halogen, C₁-C₆-alkyl, C₁-C₆-haloalkyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, C₁-C₆-alkylene and oxy-C₁-C₃-alkylenoxy;
R¹ and/or R² can carry one to four identical or different R^{a} groups:
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di(C₁-C₆-alkyl)amino, C₂-C₆-alkenyl, C₂-C₆-alkenyloxy, C₃-C₆-alkynyloxy, oxy-C₁-C₃-alkylenoxy, C₃-C₆-cycloalkyl, phenyl, naphthyl or five- or six-membered saturated, partially unsaturated or aromatic heterocycle comprising one to four heteroatoms from the group consisting of O, N and S,
these aliphatic, alicyclic or aromatic groups for their part being able to be partially or completely halogenated;
X is cyano, C₁-C₄-alkoxy, C₃-C₄-alkenyloxy, C₁-C₂-haloalkoxy or C₃-C₄-halo-alkenyloxy.

2. The compound of the formula I according to claim 1, wherein X represents cyano.

3. The compound of the formula I according to claim 1, wherein X represents methoxy.

4. The compound of the formula I according to claim 1, wherein R¹ and R² have the following meanings:
R¹ is CH(CH₃)—CH₂CH₃, CH(CH₃)—CH(CH₃)₂, CH(CH₃)—C(CH₃)₃, CH(CH₃)—CF₃, CH₂C(CH₃)═CH₂, CH₂CH═CH₂, cyclopentyl or cyclohexyl;
R² is hydrogen or methyl; or
R¹ and R² together form —(CH₂)₂CH(CH₃)(CH₂)₂—, —(CH₂)₂CH(CF₃)(CH₂)₂— or —(CH₂)₂O(CH₂)₂—.

5. A compound of the formula I.1: 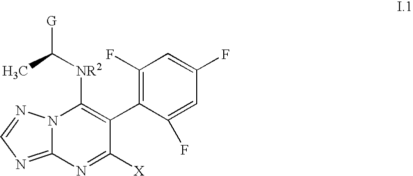
wherein
G represents C₂-C₆-alkyl, C₁-C₄-alkoxymethyl or C₃-C₆-cycloalkyl;
R² represents hydrogen or methyl; and
X represents cyano, methoxy or ethoxy.

6. A compound of the formula I.2: 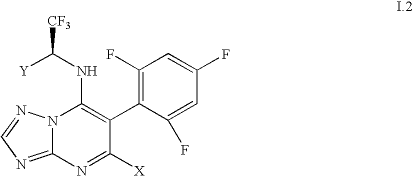
wherein Y is hydrogen or C₁-C₄-alkyl and X is cyano, methoxy or ethoxy.

7. A compound of the formula I.3: 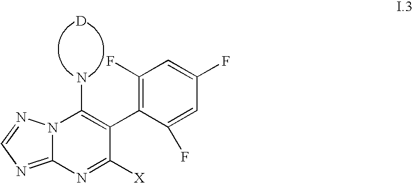
wherein
D together with the nitrogen atom, forms a five- or six-membered heterocyclyl or heteroaryl which is bonded via N and can comprise an additional heteroatom from the group consisting of O, N and S as ring member and/or can carry one or more substituents from the group consisting of halogen, C₁-C₄-alkyl, C₁-C₄-alkoxy and C₁-C₂-haloalkyl; and
X represents cyano, methoxy or ethoxy.

8. A process for the preparation of the compound of the formula I according to claim 1, by reaction of 5-halo-6-(2,4,6-trifluorophenyl)triazolopyrimidines of the formula II 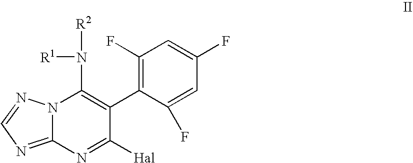
in which Hal is a halogen atom, with compounds of the formula III
M-X III
in which M is an ammonium, tetraalkylammonium or alkali metal or alkaline earth metal cation and X has the meaning according to claim 1.

9. A preparation comprising a solid or liquid carrier and a compound of the formula I according to claim 1.

10. A seed, comprising a compound of the formula I according to claim 1 in an amount of 1 to 1000 g/100 kg.

11. A process for the control of harmful phytopathogenic fungi, which comprises treating the fungi or the materials, plants, ground or seeds to be protected from fungal attack with an effective amount of a compound of the formula I according to claim 1.

12. The compound of the formula I according to claim 2, wherein R¹ and R² have the following meanings:
R¹ is CH(CH₃)—CH₂CH₃, CH(CH₃)—CH(CH₃)₂, CH(CH₃)—C(CH₃)₃, CH(CH₃)—CF₃, CH₂C(CH₃)═CH₂, CH₂CH═CH₂, cyclopentyl or cyclohexyl;
R² is hydrogen or methyl; or
R¹ and R² together form —(CH₂)₂CH(CH₃)(CH₂)₂—, —(CH₂)₂CH(CF₃)(CH₂)₂— or —(CH₂)₂O(CH₂)₂—.

13. The compound of the formula I according to claim 3, wherein R¹ and R² have the following meanings:
R¹ is CH(CH₃)—CH₂CH₃, CH(CH₃)—CH(CH₃)₂, CH(CH₃)—C(CH₃)₃, CH(CH₃)—CF₃, CH₂C(CH₃)═CH₂, CH₂CH═CH₂, cyclopentyl or cyclohexyl;
R² is hydrogen or methyl; or
R¹ and R² together form —(CH₂)₂CH(CH₃)(CH₂)₂—, —(CH₂)₂CH(CF₃)(CH₂)₂— or —(CH₂)₂O(CH₂)₂—.
